(19) United States
(12) Patent Application Publication
(10) Pub. No.: US 2013/0211203 A1
(43) Pub. Date: Aug. 15, 2013

(54) Surgical Retractor

(71) Applicants: Camil Sader, Pompano Beach, FL (US); Mohammed Abdullah, Deerfield Beach, FL (US); Ramon Jadra, Reistertown, MD (US)

(72) Inventors: Camil Sader, Pompano Beach, FL (US); Mohammed Abdullah, Deerfield Beach, FL (US); Ramon Jadra, Reistertown, MD (US)

(21) Appl. No.: 13/372,558

(22) Filed: Feb. 14, 2012

(51) Int. Cl. A61B 1/32

(52) U.S. Cl. 600/216

(57) ABSTRACT

A surgical retractor designed for wounds and incisions located on curved areas of the body. The clamp assembly comprising the present surgical retractor comprises multiple hinges and other types of movable joints allowing it to be adjusted to any number of positions in order to conform to various areas of the body as well as wounds or incisions of different sizes. In particular, the clamp assembly can comprise hinges located in the middle of at least two of its sides allowing it to be bent into multiple angles both above and below the flat horizontal position allowed by nearly all existing surgical retractors. Multiple retracting hook assemblies can be mounted at various positions along the sides of the clamp assembly allowing the hook comprising each to hold tissue in a variety of positions as needed.

### FIELD OF THE INVENTION

The present device is a surgical retractor capable of self-retracting wounds and incisions, particularly those located on curved surfaces of the body. This device can allow greater access to areas exposed during surgeries and can allow retractions to occur at nearly any position desired by the health practitioner. The present device is designed for use with wounds located on parts of the anatomy where traditional retractors struggle to function properly.

### BACKGROUND

Wound retractors have long been used to hold open a patient's skin surrounding an incision, thus allowing the surgeon to work internally on a patient. Surgeons and support staff commonly use self-retracting wound retractors to keep their hands free to perform other necessary tasks. These retractors can be adjusted into a proper position and then locked in place to maintain that position. However, existing self-retracting wound retractor models often lack the stability required to maintain a surgical site sufficiently free from objects that can obstruct the view of a patient's internal body cavity. Additionally, retractors that are capable of holding a wound open can cause a significant amount of damage to the surrounding tissue.

In the past, a secure mount was achieved through the use of an attachment to a steady object, such as a bed post. However, any extension from the patient's body to a point outside of the body can both pose a hazard if bumped or jostled and can be an obstruction to a surgeon attempting to perform a surgery. Additionally, if the attachment mechanism is located below the edge of the bed, it can be in close proximity to areas that are not sterile, which could lead to contamination of the surgical area and infection of the wound site.

Other solutions that have been used to maintain proper retraction of an incision involve the use of retractors or objects that can be inserted into the body cavity. Such retractors retract a surgical opening through the use of tension created when opposite sides of a wound are pulled and held apart. These retractors tend to be flat and are not capable of conforming to a curved surface. The use of such flat retractors can create uneven or unnecessary forces on the skin surrounding the incision, which can cause damage to the patient's body. These designs can be utilized at locations on the body where substantial space surrounding the wound is available. However, they cannot be used in locations adjacent to a curved surface, such as the groin or neck because they require a flat area in order to create the correct tensioning forces.

What is needed is a self-retracting wound retractor for use with incisions located on curved areas of the body such as the groin, neck, knee, or elbow that is capable of maintaining a constant cavity size while causing minimal damage to the tissue surrounding the incision.

### SUMMARY OF THE INVENTION

It is an aspect of the present device to provide a self-retracting wound retractor for use with incisions located on curved areas of the body, such as the groin, neck, knee, or elbow that is capable of maintaining a constant cavity size while causing minimal damage to the tissue surrounding the incision.

The above aspects can be obtained by a surgical retractor comprising: a clamp assembly comprising four sides, each side having a first end and a second end, wherein a first end of a first side is configured to be movably connected to a first end of a third side by a first hinged joint, a second end of the first side is configured to be movably connected to a first end of a fourth side at a first adjustable point along the fourth side and a second end of the fourth side is configured to be movably connected to a second end of a second side at a second adjustable point along the fourth side and a first end of the second side is configured to be movably connected to a second end of the third side by a second hinged joint; and two or more retracting hook assemblies, each hook assembly comprising an arm having a first end and a second end, wherein a hook is connected to the first end, and an attachment block connected to the arm capable of securely holding the arm in a particular position and also configured to connect the retracting hook assembly to one of the four sides of the clamp assembly.

The above aspects can also be obtained by a surgical retractor comprising: a clamp assembly comprising four sides, each side having a first end and a second end, wherein a first end of a first side is configured to be movably connected to a first end of a third side by a first hinged joint, a second end of the first side is configured to be movably connected to a first end of a fourth side at a first adjustable point along the fourth side and a second end of the fourth side is configured to be movably connected to a second end of a second side at a second adjustable point along the fourth side and a first end of the second side is configured to be movably connected to a second end of the third side by a second hinged joint; a first screw clamp configured to movably connect a second end of the first side to a first adjustable point along a fourth side and a second screw clamp configured to movably connect a second end of the second side to a second adjustable point along the fourth side; a second side hinge separating the second side into a first section of the second side and a second section of the second side, wherein the second side hinge is configured to permit the first section of the second side and the second section of the second side to bend therebetween in a direction substantially perpendicular to a length of the second side; and a first side hinge separating the first side into a first section of the first side and a second section of the first side, wherein the first side hinge is configured to permit the first section of the first side and the second section of the first side to bend therebetween in a direction substantially perpendicular to a length of the first side.

The above aspects can also be obtained by a method for using a surgical retractor, the method comprising: providing a surgical retractor comprising a clamp assembly comprising four sides, each side having a first end and a second end, wherein a first end of a first side is configured to be movably connected to a first end of a third side by a first hinged joint, a second end of the first side is configured to be movably connected to a first end of a fourth side at a first adjustable point along the fourth side and a second end of the fourth side is configured to be movably connected to a second end of a second side at a second adjustable point along the fourth side and a first end of the second side is configured to be movably connected to a second end of the third side by a second hinged joint; a first screw clamp configured to movably connect a second end of the first side to a first adjustable point along a fourth side and a second screw clamp configured to movably connect a second end of the second side to a second adjustable point along the fourth side; a second side hinge separating the second side into a first section of the second side and a second section of the second side, wherein the second side hinge is configured to permit the first section of the second side and the second section of the second side to bend therebetween in a direction substantially perpendicular to a length of the second side; a first side hinge separating the first side into a first section of the first side and a second section of the first side, wherein the first side hinge is configured to permit the first section of the first side and the second section of the first side to bend therebetween in a direction substantially perpendicular to a length of the first side; and at least two retracting hook assemblies configured to removably connect to the clamp assembly; and making an incision in the skin covering a surgical site; placing the surgical retractor over the incision and bending the first side hinge and second side hinge so that the first side and second side of the clamp assembly conforms to the shape of the surgical site; connecting at least one retracting hook assembly on the first side of the clamp assembly and at least one retracting hook assembly on the second side of the clamp assembly; connecting the hooks comprising the retracting hook assemblies inside the incision and into contact with the skin surrounding the incision; and opening the incision by pulling the first and second sides apart from each other about their respective first and second hinged connections and securing each side in place by connecting it to a desired point along the fourth side.

### BRIEF DESCRIPTION OF THE DRAWINGS

Further features and advantages of the present device, as well as the structure and operation of various embodiments of the present device, will become apparent and more readily appreciated from the following description of the preferred embodiments, taken in conjunction with the accompanying drawings of which:

FIG. 1 is a top view of a clamp assembly for a surgical retractor according to an embodiment;

FIG. 2 is a side view of a clamp assembly for a surgical retractor bent at an angle according to an embodiment;

FIG. 3 is a side view of a retractor element for a surgical retractor according to an embodiment;

FIG. 4 is a front view of a retractor element for a surgical retractor according to an embodiment;

FIG. 5 is a top view of a surgical retractor comprising four retracting hook assembly's connected to a clamp assembly according to an embodiment;

FIG. 6 is a side view of a surgical retractor comprising four retracting hook assembly's bent at an angle according to an embodiment;

FIG. 7 is a top view of a surgical retractor in use at a surgical site holding tissue away from a body cavity where an operation is being performed according to an embodiment;

FIG. 8 is a side view of a surgical retractor in use at a surgical site holding tissue away from a body cavity where an operation is being performed according to an embodiment; and

FIG. 9 is a view of three different locations where a surgery on a body can be benefited by the use of the present surgical retractor according to an embodiment.

### DETAILED DESCRIPTION

This description of the exemplary embodiments is intended to be read in connection with the accompanying drawings, which are to be considered part of the entire written description. In the description, relative terms such as “lower,” “upper,” “horizontal,” “vertical,”, “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

Reference will now be made in detail to the presently preferred embodiments of the invention, examples of which are illustrated in the accompanying drawings, wherein like reference numerals refer to like elements throughout.

The present surgical retractor is designed for use with operations, particularly those involving curved body surfaces, such as the groin and neck. Small versions of the present surgical retractor can be used for small incisions and larger versions can be used for larger incisions. Some common procedures performed in these areas include: inguinal hernia repairs; femoral hernia repairs; femoral-femoral bypass; femoral-popliteal bypass; femoral-distal bypass; axillary bifemoral bypass; and carotid endarterectomies. These procedures require oblique incisions in the groin or vertical incisions in the groin or neck. Neither of these locations offer a surgeon sufficient workspace and much of the space that is available is typically occupied by the wound retractors. Additionally, these locations are curved and most retractors are primarily suited for use on flat surfaces. Use of such retractors at these locations can cause damage to the patient's tissue surrounding the surgical site.

In an embodiment, the present wound retractor can comprise a clamp assembly having four sides roughly forming a rectangle, within which a surgery can be performed. Two of the opposing sides, a first side and a second side, can be curved, or arc-shaped, to create a larger opening in the middle than at the ends. Each of these opposing sides can be attached through the use of a hinge to a straight segment, or third side, at one end and adjustably attached at a second end to a fourth side, located opposite the third side. Retracting elements comprising a hook-like shape at one end can be attached to any or all of the sides of the clamp assembly allowing it to sufficiently hold open a wound or incision. The hook portion of the retracting element can be used to pull the tissue back towards the sides of the clamp assembly. The tension created by pulling opposing sides of the opening can thus be used to hold the retractor in place around it. Any number of opposing retractor elements can be placed onto the clamp assembly, with four being a preferable number for smaller incisions and six being a preferable number for larger incisions. However, the surgeon would have the option of using any number of retractor elements that could fit onto the clamp assembly.

Many operations require the wound to be closed in a gradual fashion, allowing sutures to be inserted as the wound is slowly closed. The present retractor can comprise hinges at both a first end of a third side and a second end of a third side. These hinges can allow the first side and second side of the retractor to be gradually adjusted either towards the center of the incision or away from it through the use of two releasing mechanisms located at both the second end of the first side and the second end of the second side. These releasing mechanisms can be used to removably connect the second end of the first side and the second end of the second side to adjustable points along the fourth side, thus allowing the retractor to be easily manipulated by a surgeon or a single assistant.

The present wound retractor can also comprise a first side hinge and a second side hinge, which can allow the first side and second side to bend in their respective middles in directions substantially perpendicular to their respective lengths. The angle of this bend can be adjusted so that the retractor can fit the contour of a particular curved surface of a patient's body, thus preventing unwanted vertical forces that can cause unnecessary tissue damage.

FIG. 1 is a top view of a clamp assembly 100 comprising a surgical retractor according to an embodiment.

The clamp assembly 100, as depicted in FIG. 1, can be comprised of four sides roughly forming a rectangle 150 within which a surgery can be performed near the rectangle's center 101. A third side 102 can have a first end 103 and a second end 104. The second end 104 of the third side 102 can be movably connected to a first end 105 of a second side 106 by a first hinged joint 107 at a first angle 108. The first end 103 of the third side 102 can be movably connected to a first end 109 of a first side 110 by a second hinged joint 111 at a second angle 112.

The second side 106 and first side 110 can be approximately the same size and shape, or as in the case of the embodiment depicted in FIG. 1, the two can be minor images of each other. In a preferred embodiment, both the second side 106 and the first side 110 can be arc-shaped, wherein each arc bows away from the rectangle's center 101. The second side 106 can comprise a second side hinge 113 separating the second side 106 into a first section 126 of the second side 106 and a second section 136 of the second side 106. The second side hinge 113 can be configured to allow the second side 106 to bend therebetween in a direction substantially perpendicular to a length of the second side 106. Likewise, the first side 110 can comprise a first side hinge 123 separating the first side 110 into a first section 130 of the first side 110 and a second section 140 of the first side 110. The first side hinge 123 can be configured to to allow the first side 110 to bend therebetween in a direction substantially perpendicular to a length of the first side. The first side hinge 113 and second side hinge 123 can allow the first side 110 and second side 106 to conform to the contour of a surgical site.

A fourth side 114 can complete the roughly rectangular shape 150 encompassed within the clamp assembly 100 by connecting to the second end 117 of the second side 106 and to the second end 119 of the first side 110. The fourth side can comprise a first end 115 and a second end 116. The second end 117 of the second side 106 can be movably connected at or near the first end 115 of the fourth side 114 using a first screw clamp 118, wherein a first screw (not pictured) comprising the first screw clamp 118 can be threaded through a first hole (not pictured) located at the second end 117 of the second side 106. This can allow the first screw to be tightened allowing the attachment of the second end 117 of the second side 106 to various points along the length of the fourth side 114. This movement along the length of the fourth side 114 can be facilitated by the first hinged joint 107 where the first end 105 of the second side 106 connects to the first end 104 of the third side 102. In a preferred embodiment, the fourth side 114 can be flat or have at least one flat side against which the first screw can be tightened, thus securing the second side 106 to the fourth side 114. Likewise, the second end 119 of the first side 110 can be movably connected to the second end 116 of the fourth side 114 using a second screw clamp 120, wherein a second screw (not pictured) comprising the second screw clamp 120 can be threaded through a second hole (not pictured) located at the second end 119 of the first side 110.

The clamp assembly 100 can be made substantially of stainless steel, plastic, or any other suitable material known in the art of surgical instruments. Furthermore, although the clamp assembly 100, as depicted in FIG. 1, is roughly rectangular in shape, the present surgical retractor could be configured to comprise clamp assemblies of various other shapes.

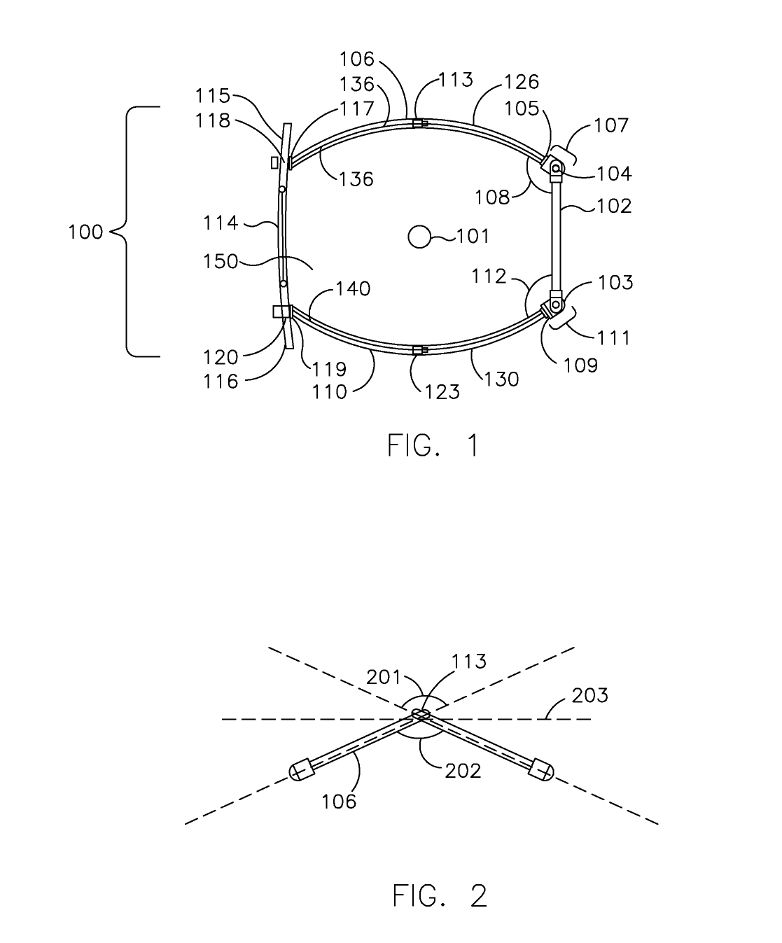

FIG. 2 is a side view of a clamp assembly for a surgical retractor bent at an angle 202 according to an embodiment.

The second side 106 can comprise a second side hinge 113 separating the second side 106 into a first section of the second side 126 and a second section of the second side 136. Likewise, the first side 110 can comprise a first side hinge 123 separating the first side 110 into a first section of the first side 130 and a second section of the first side 140. The first side hinge 123 and second side hinge 113 can allow the clamp assembly 100 to form obtuse angles both above 201 and below 202 the flat, horizontal 203 position to which most surgical retractors are confined. This feature can allow the clamp assembly 100 to conform to the shape of a patient's body, especially in locations, such as the neck, groin and medial knee areas, which do not provide large flat surfaces upon which to operate.

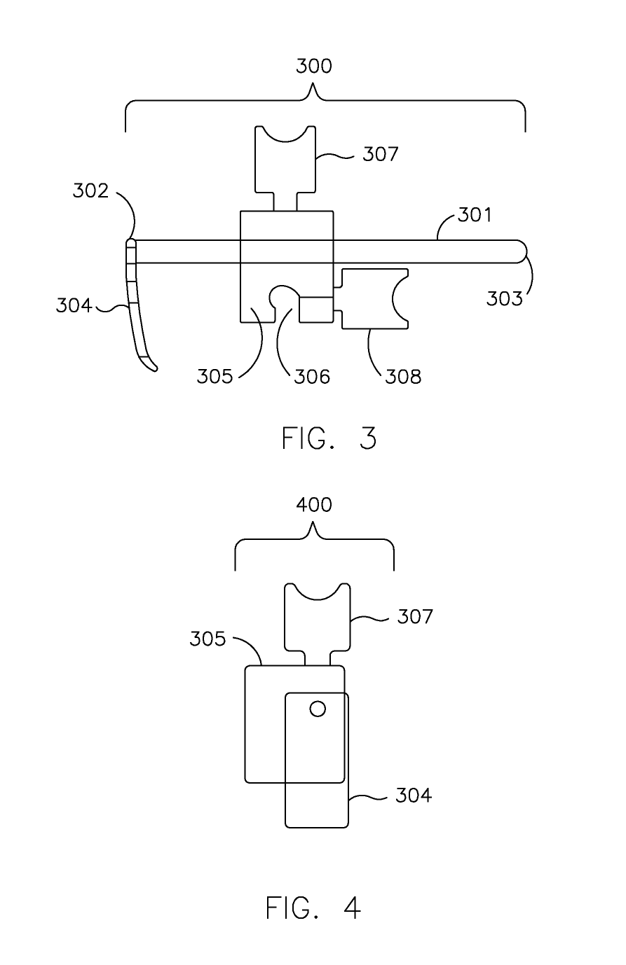

FIG. 3 is a perspective side view of a retracting hook assembly 300 comprising a surgical retractor according to an embodiment.

The present surgical retractor can comprise one or more retracting hook assemblies 300 which can be movably connected to the clamp assembly 100. Each retracting hook assembly 300 can comprise an arm 301 having an elongated, pin-like shape comprising a first end 302 and a second end 303. The arm 301 can be connected to a hook 304 at its first end 302. In an embodiment, this hook 304 can be connected perpendicularly to the arm 301. Additionally, the hook 304 can comprise a flat surface allowing it to hold tissue (not pictured) in place without damaging it. However, the retracting hooks can be of various dimensions, which can be better suited to particular needs and the present surgical retractor should be configured to be used with any retracting hook.

The retracting hook assembly 300 can also comprise an attachment block 305, which can be used to secure the retracting hook assembly 300 to the clamp assembly 100. The attachment block 305 can comprise a first rounded slot (not pictured) and a second rounded slot 306. In a preferred embodiment, the first rounded slot can be located at a first side of the attachment block 305 and the second rounded slot 306 can be located at a second side of the attachment block 305. The first rounded slot can also be located at an angle perpendicular to the second rounded slot 306. The attachment block 305 can also comprise a first thumb screw 307, passing through a first threaded hole, which can connect attachment block 305 to any point along the arm 301, which can pass through the first rounded slot, by tightening the first thumb screw 307 against the arm 301. A second thumb screw 308, passing through a second threaded hole (not pictured) into the second rounded slot 306, can connect the attachment block 305 to any point along the clamp assembly 100 by tightening the second thumb screw 308 against any side of the clamp assembly 100.

The first rounded slot can be of a size and shape sufficient to allow the length of the arm 301 to slide back and forth through it and to be turned radially within it. The first thumb screw 307 can then be tightened against the arm 301 in order to hold it and the hook 304 in any desired position. Likewise, the second rounded slot 306 can fit over any point along the length of one of the four sides comprising the clamp assembly 100 so as to allow the retracting hook assembly 300 to rotate freely around any of the four sides. The second thumb screw 308 can be tightened against any one of the clamp assembly's 100 sides in order to securely connect it in a desired position. The second rounded slot 306 can have an opening located on the bottom of the retractor element that can allow the retractor element to be fully removed from the clamp assembly. The second thumb screw 308 connecting the retracting hook assembly 300 to the clamp assembly 100 can be loosened slightly in order to allow the retracting hook assembly 300 to be repositioned without removing it from the clamp assembly 100 completely.

FIG. 4 is a front view of a retracting hook assembly 400 according to an embodiment.

In an embodiment, a flat and wide hook 304, as depicted in FIG. 4, can comprise the retracting hook assembly 400. Although the hook 304 in this figure is shown in a position parallel to the attachment block 305, it can be rotated into any position and locked into that position by tightening the first thumb screw 307 against the arm 301 (not pictured).

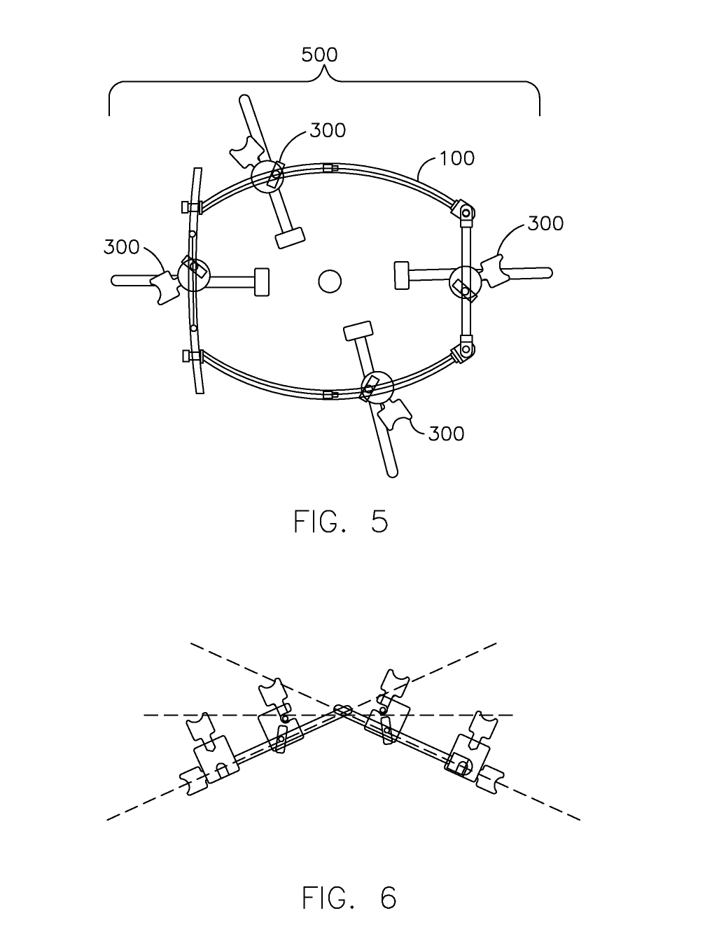

FIG. 5 is a top view of a surgical retractor 500 comprising four retracting hook assemblies 300 connected to a clamp assembly 100 according to an embodiment.

One or more retracting hook assemblies 300 can be connected to each of the four sides of the clamp assembly 100 to complete the surgical retractor 500 in the desired conformation. Each side of the clamp assembly 100 can comprise any number of retracting hook assemblies 300 ranging from one to as many as can be connected to the length of a particular side. However, practically speaking, only one or two retracting hook assemblies 300 can typically be used per side of the clamp assembly 100. In a preferred embodiment, the retracting hook assemblies 300 can be located at opposing positions along the clamp assembly 100 in order to provide even tension to hold open a wound or incision.

FIG. 6 is a side view of a surgical retractor 500 comprising four retracting hook assemblies 300 connected to a clamp assembly 100 according to an embodiment.

As described above, the present clamp assembly 100 can be bent at the first side hinge at an angle substantially perpendicular to the length of the first side and at the second side hinge at an angle perpendicular to the length of the second side, thus providing the best possible contact between the hooks 304 (not pictured in FIG. 3) and the tissue to be held by each hook 304. Generally, this angle will correlate to the curvature of the part of the body where the surgical retractor is being used.

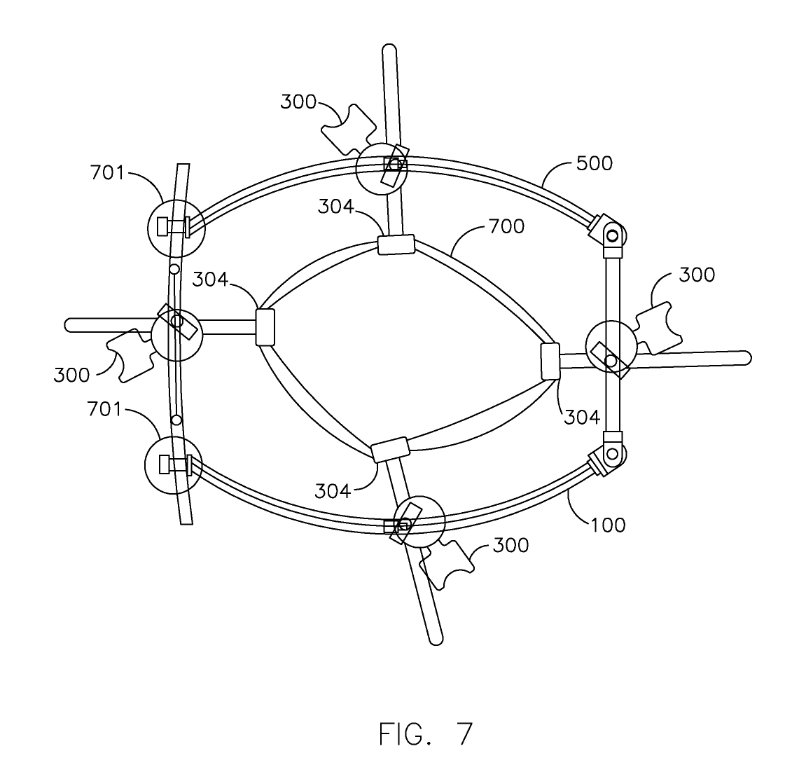

FIG. 7 is a top view of a surgical retractor 500, comprising four retracting hook assemblies 300 connected to a clamp assembly 100, in use at a surgical site holding tissue away from the body cavity 700 where an operation is being performed.

The surgical retractor 500 can be assembled from a kit prior to use, which can require a minimum of two retracting hook assemblies 300 connected to the clamp assembly 100. The various screws and thumbscrews comprising the clamp assembly 100 and each retracting hook assembly 300 respectively can each be loosened to allow each of the surgical retractor's 500 movable connections to be adjusted into an optimal position for the particular surgery being performed. The hooks 304 comprising the retracting hook assemblies 300 can be used to grasp the edge of a wound or incision and can be adjusted outward from the center of the wound or incision stretching its edges apart, exposing the internal parts of the body being worked upon within the body cavity 700. Additional retractor elements can be added at any time during the surgery to ensure that the cavity 700 is held open sufficiently or to position different layers of tissue.

After the procedure has been completed, the wound or incision can gradually be closed by adjusting the hooks 304 incrementally toward the center of the wound or incision. These hooks 304 can each be adjusted toward the center of the body cavity 700 in two ways. First, the arms 301 comprising each retracting hook assembly 300 can be adjusted inward by sliding their positions inward through the first rounded slot and held in place using the first thumb screw 307. Second, a more preferable method for closing the wound or incision can involve the adjustment of the sliding joints 701 connecting the fourth side 114 to both the second side 106 and the third side 110 using the first screw clamp 118 and second screw clamp 120 respectively. This can allow a single user to simultaneously adjust the tissue position of the body cavity 700 along an entire side of the clamp assembly 100, upon which more than one retracting hook assembly 300 can be mounted.

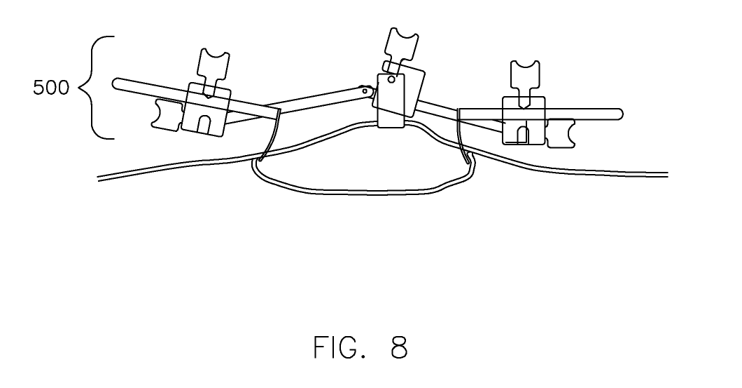

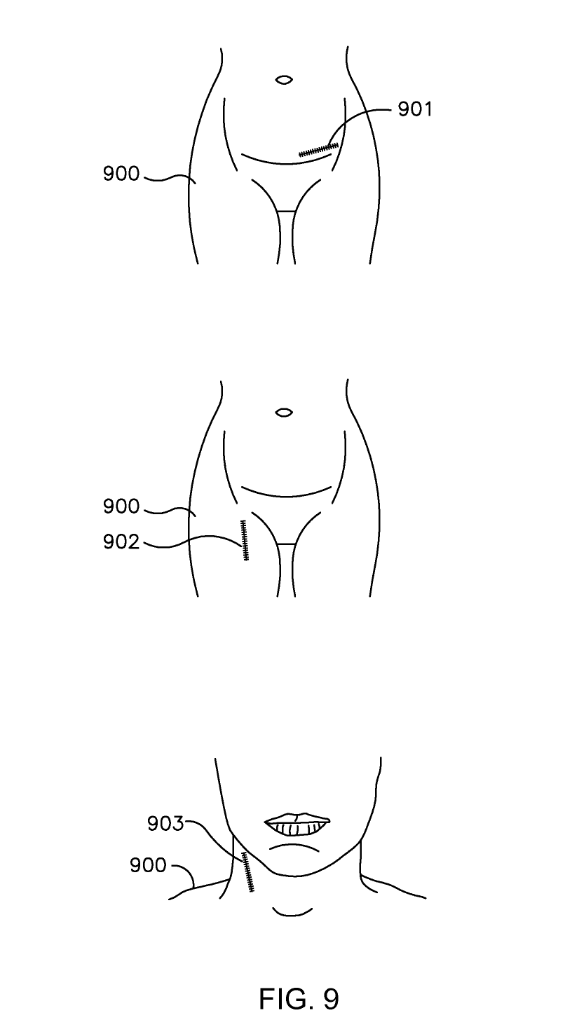

FIG. 8 is a side view of a surgical retractor, comprising three retracting hook assemblies 300 connected to a clamp assembly 100, in use at a surgical site holding tissue away from the body cavity where an operation is being performed.

The hinges 113 located in the center of the second side 106 and third side 110 of the clamp assembly 100 can be used to maintain a proper depth of the hooks 304 in the wound or incision. When the shape of the clamp assembly 100 is not capable of adjusting to fit the curvature of the area of the body being worked upon, some edges of the wound may be subjected to vertical forces, which could cause unnecessary damage to the tissues affected. The ability to bend in the clamp assembly 100 can allow each clamp to be at approximately the same height relative to the patient and the hooks to be at the same depth, which can prevent these vertical forces from occurring.

FIG. 9 is a depiction of three segments of a human body 900 indicating three different locations where a surgery could be benefited by the use of the present surgical retractor.

The present surgical retractor can be used for almost any surgical procedure. However, it is specially designed for use in locations that are not flat. These non-uniform locations can include, but are not limited to wounds that are located in close proximity to the groin 901 and 902 and neck 903. Specifically, the present device can be used to perform procedures involving the anterior and lateral neck, as well as lingual, femoral, upper and lower popliteal areas of the body. The human anatomy creates tight spaces and curves in these locations, which makes the use of ordinary surgical retractors almost, if not completely impossible. The present invention is capable of being used in these locations due to the presence of the hinges on the sides, which allow the retractor to conform to the curves of the body. Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. A surgical retractor comprising:
a clamp assembly comprising four sides, each side having a first end and a second end, wherein a first end of a first side is configured to be movably connected to a first end of a third side by a first hinged joint, a second end of the first side is configured to be movably connected to a first end of a fourth side at a first adjustable point along the fourth side and a second end of the fourth side is configured to be movably connected to a second end of a second side at a second adjustable point along the fourth side and a first end of the second side is configured to be movably connected to a second end of the third side by a second hinged joint; and
two or more retracting hook assemblies, each hook assembly comprising an arm having a first end and a second end, wherein a hook is connected to the first end, and an attachment block connected to the arm capable of securely holding the arm in a particular position and also configured to connect the retracting hook assembly to one of the four sides of the clamp assembly.

2. The surgical retractor as described in claim 1, further comprising a second side hinge separating the second side into a first section of the second side and a second section of the second side, wherein the second side hinge is configured to permit the first section of the second side and the second section of the second side to bend therebetween in a direction substantially perpendicular to a length of the second side; and

3. The surgical retractor as described in claim 2, further comprising a first side hinge separating the first side into a first section of the first side and a second section of the first side, wherein the first side hinge is configured to permit the first section of the first side and the second section of the first side to bend therebetween in a direction substantially perpendicular to a length of the first side.

4. The surgical retractor as recited in claim 1, wherein the retractor is not assembled and is in kit form.

5. The surgical retractor as recited in claim 1, wherein the retractor is assembled around a patient.

6. The surgical retractor as described in claim 1 wherein the retracting hook assemblies further comprise thumb screws configured to removably connect the arm to the attachment block.

7. The surgical retractor as described in claim 1 wherein the retracting hook assemblies further comprise thumb screws configured to removably connect the attachment block to one side of the clamp assembly.

8. The surgical retractor as described in claim 1 wherein the second end of the second side is attached to the first adjustable point with a first screw clamp and the second end of the first side is attached to the second adjustable point with a second screw clamp.

9. The surgical retractor as described in claim 1 wherein the second side and the first side are both arc-shaped wherein the respective arcs bow away from a center of the surgical retractor.

10. A surgical retractor comprising:
a clamp assembly comprising four sides, each side having a first end and a second end, wherein a first end of a first side is configured to be movably connected to a first end of a third side by a first hinged joint, a second end of the first side is configured to be movably connected to a first end of a fourth side at a first adjustable point along the fourth side and a second end of the fourth side is configured to be movably connected to a second end of a second side at a second adjustable point along the fourth side and a first end of the second side is configured to be movably connected to a second end of the third side by a second hinged joint;
a first screw clamp configured to movably connect a second end of the first side to a first adjustable point along a fourth side and a second screw clamp configured to movably connect a second end of the second side to a second adjustable point along the fourth side;
a second side hinge separating the second side into a first section of the second side and a second section of the second side, wherein the second side hinge is configured to permit the first section of the second side and the second section of the second side to bend therebetween in a direction substantially perpendicular to a length of the second side; and
a first side hinge separating the first side into a first section of the first side and a second section of the first side, wherein the first side hinge is configured to permit the first section of the first side and the second section of the first side to bend therebetween in a direction substantially perpendicular to a length of the first side.

11. The retractor as recited in claim 10, further comprising:
two or more retracting hook assemblies, each hook assembly comprising:
an arm having a first end and a second end, wherein a hook is connected to the first end; and
an attachment block is connected to the arm.

12. The retractor as recited in claim 10, wherein the retractor is not assembled and is in kit form.

13. The retractor as recited in claim 10, wherein the retractor is assembled around a patient.

14. The retractor as recited in claim 11, wherein the retractor is not assembled and is in kit form.

15. The retractor as recited in claim 11, wherein the retractor is assembled around a patient.

16. A method for using a surgical retractor comprising:
providing a surgical retractor comprising a clamp assembly comprising four sides, each side having a first end and a second end, wherein a first end of a first side is configured to be movably connected to a first end of a third side by a first hinged joint, a second end of the first side is configured to be movably connected to a first end of a fourth side at a first adjustable point along the fourth side and a second end of the fourth side is configured to be movably connected to a second end of a second side at a second adjustable point along the fourth side and a first end of the second side is configured to be movably connected to a second end of the third side by a second hinged joint; a first screw clamp configured to movably connect a second end of the first side to a first adjustable point along a fourth side and a second screw clamp configured to movably connect a second end of the second side to a second adjustable point along the fourth side; a second side hinge separating the second side into a first section of the second side and a second section of the second side, wherein the second side hinge is configured to permit the first section of the second side and the second section of the second side to bend therebetween in a direction substantially perpendicular to a length of the second side; a first side hinge separating the first side into a first section of the first side and a second section of the first side, wherein the first side hinge is configured to permit the first section of the first side and the second section of the first side to bend therebetween in a direction substantially perpendicular to a length of the first side; and at least two retracting hook assemblies configured to removably connect to the clamp assembly; and
making an incision in the skin covering a surgical site;
placing the surgical retractor over the incision and bending the first side hinge and second side hinge so that the first side and second side of the clamp assembly conforms to the shape of the surgical site;
connecting at least one retracting hook assembly on the first side of the clamp assembly and at least one retracting hook assembly on the second side of the clamp assembly;
connecting the hooks comprising the retracting hook assemblies inside the incision and into contact with the skin surrounding the incision; and
opening the incision by pulling the first and second sides apart from each other about their respective first and second hinged connections and securing each side in place by connecting it to a desired point along the fourth side.

17. The method for using a surgical retractor as recited in claim 16 wherein the retracting hook assemblies further comprise thumb screws configured to removably connect an arm to an attachment block.

18. The method for using a surgical retractor as recited in claim 16 wherein the retracting hook assemblies further comprise thumb screws configured to removably connect the attachment block to one side of the clamp assembly.

19. The method for using a surgical retractor as recited in claim 16 wherein the second end of the first side is configured to attach to any point along the length of the fourth side with a first screw clamp and the second end of the second side is configured to attach to any point along the length of the fourth side with a second screw clamp.

20. The surgical retractor as recited in claim 10 wherein the surgical retractor is primarily comprised of stainless steel.
